SEQUENCE LISTING (23 sequences)

SEQ ID NO: 1 - DNA, 219 bp, Escherichia coli
atggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctga

SEQ ID NO: 2 - DNA, 1332 bp, Escherichia coli
atggctaaacaaccgggattagattttcaaagtgccaaaggtggcttaggcgagctgaaacgcagactgctgtttgttatcggtgcgctgattgtgttccgtattggctcttttattccgatccctggtattgatgccgctgtacttgccaaactgcttgagcaacagcgaggcaccatcattgagatgtttaacatgttctctggtggtgctctcagccgtgcttctatctttgctctggggatcatgccgtatatttcggcgtcgatcattatccagctgctgacggtggttcacccaacgttggcagaaattaagaaagaaggggagtctggtcgtcgtaagatcagccagtacacccgctacggtactctggtgctggcaatattccagtcgatcggtattgctaccggtctgccgaatatgcctggtatgcaaggcctggtgattaacccgggctttgcattctacttcaccgctgttgtaagtctggtcacaggaaccatgttcctgatgtggttgggcgaacagattactgaacgaggtatcggcaacggtatttcaatcattatcttcgccggtattgtcgcgggactcccgccagccattgcccatactatcgagcaagcgcgtcaaggcgacctgcacttcctcgtgttgctgttggttgcagtattagtatttgcagtgacgttctttgttgtatttgttgagcgtggtcaacgccgcattgtggtaaactacgcgaaacgtcagcaaggtcgtcgtgtctatgctgcacagagcacacatttaccgctgaaagtgaatatggcgggggtaatcccggcaatcttcgcttccagtattattctgttcccggcgaccatcgcgtcatggttcgggggcggtactggttggaactggctgacaacaatttcgctgtatttgcagcctgggcaaccgctttatgtgttactctatgcgtctgcaatcatcttcttctgtttcttctacacggcgttggttttcaacccgcgtgaaacagcagataacctgaagaagtccggtgcatttgtaccaggaattcgtccgggagagcaaacggcgaagtatatcgataaagtaatgacccgcctgaccctggttggtgcgctgtatattacctttatctgcctgatcccggagttcatgcgtgatgcaatgaaagtaccgttctacttcggtgggacctcactgcttatcgttgttgtcgtgattatggactttatggctcaagtgcaaactctgatgatgtccagtcagtatgagtctgcattgaagaaggcgaacctgaaaggctacggccgataa

SEQ ID NO: 3 - DNA, 72 bp, Artificial Sequence
  primer
tgcagaccttgtggcaacaatttctacaaaacacttgatactgtatgagccagccccatacgatataagttg

SEQ ID NO: 4 - DNA, 70 bp, Artificial Sequence
  primer
gtgggaataagataagttttcttgactgggaagtaaaataccgtatgcgtcatcgagacaagttacggac

SEQ ID NO: 5 - DNA, 3048 bp, Artificial Sequence
  knock-in cassette
tgcagaccttgtggcaacaatttctacaaaacacttgatactgtatgagccagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaagttcattagggctcgaagttcctatactttctagagaataggaacttctcattaatcctgttctcttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcattgtcaacacgtgctgaagttcctatactttctagagaataggaacttccgccctttagtgagggttaattgtctaagaaaccattattatcatgacattaacctataaaaataggcgtatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgacgcatacggtattttacttcccagtcaagaaaacttatcttattcccac

SEQ ID NO: 6 - DNA, 68 bp, Artificial Sequence
  primer
atgataattattgtttgctcatactgtaaacaagttgtgcagtatatctactatagggctcgaggaag

SEQ ID NO: 7 - DNA, 73 bp, Artificial Sequence
  primer
gtgatatgtatgaagtactttggaagtataagtccgtaacttgtctcgatgttaattaagtttaaacgcggcc

SEQ ID NO: 8 - DNA, 1034 bp, Artificial Sequence
  knock-in cassette
atgataattattgtttgctcatactgtaaacaagttgtgcagtatatctactatagggctcgaggaagttcctatactttctagagaataggaacttccgcgccgcacacaaaaaccaacacacagatcatgaaaataaagctcttttattggtaccgaattcgccagggagctctcagacgtcgcttggtcggtctttattcgaaccccagagtcccgcttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatggtttagttcctcaccttgtcgtattatactatgccgatatactatgccgatgattaattgtcaacacgtgctgctgcagcggccgcgtttaaacttaattaacatcgagacaagttacggacttatacttccaaagtacttcatacatatcac

SEQ ID NO: 9 - DNA, 69 bp, Artificial Sequence
  primer
ggctaagccgcaccaaaagcgcaacgtataagggagcggtgagataaacgtattcgaaccccagagtcc

SEQ ID NO: 10 - DNA, 71 bp, Artificial Sequence
  primer
ctcattaccccgctgccgataaggaatttttcgcgtcaggtaacgcccataattaaccctcactaaagggc

SEQ ID NO: 11 - DNA, 1577 bp, Artificial Sequence
  knock-in cassette
ggctaagccgcaccaaaagcgcaacgtataagggagcggtgagataaacgtattcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgatcgtctgttgtgcccagtcatagccgaatagcctctccacccaagcggccggagaacctgcgtgcaatccatcttgttcaatggccgatcccatggtttagttcctcaccttgtcgtattatactatgccgatatactatgccgatgattaattgtcaacacgtgctgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtggaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatgaagttcctatactttctagagaataggaacttcgcggccgccctttagtgagggttaattatgggcgttacctgacgcgaaaaattccttatcggcagcggggtaatgag

SEQ ID NO: 12 - DNA, 5746 bp, Artificial Sequence
  plasmid
ccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcgr

SEQ ID NO: 13 - DNA, 68 bp, Artificial Sequence
  primer
gtttttttgtttgcaagcagcagattacgcgcagaaaaaaaggatctcaacggagtaatgtgccgaac

SEQ ID NO: 14 - DNA, 77 bp, Artificial Sequence
  primer
gtttatttttctaaatacattcaaatatgtatccgctcatgaattaattcgcggccgcttacgccccgccctgccac

SEQ ID NO: 15 - DNA, 1327 bp, Artificial Sequence
  expression cassette
gtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgaattaattcatgagcggatacatatttgaatgtatttagaaaaataaac

SEQ ID NO: 16 - DNA, 6036 bp, Artificial Sequence
  plasmid
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 17 - DNA, 5289 bp, Artificial Sequence
  plasmid
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 18 - DNA, 3746 bp, Artificial Sequence
  knock-in cassette
cagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggctaaacaaccgggattagattttcaaagtgccaaaggtggcttaggcgagctgaaacgcagactgctgtttgttatcggtgcgctgattgtgttccgtattggctcttttattccgatccctggtattgatgccgctgtacttgccaaactgcttgagcaacagcgaggcaccatcattgagatgtttaacatgttctctggtggtgctctcagccgtgcttctatctttgctctggggatcatgccgtatatttcggcgtcgatcattatccagctgctgacggtggttcacccaacgttggcagaaattaagaaagaaggggagtctggtcgtcgtaagatcagccagtacacccgctacggtactctggtgctggcaatattccagtcgatcggtattgctaccggtctgccgaatatgcctggtatgcaaggcctggtgattaacccgggctttgcattctacttcaccgctgttgtaagtctggtcacaggaaccatgttcctgatgtggttgggcgaacagattactgaacgaggtatcggcaacggtatttcaatcattatcttcgccggtattgtcgcgggactcccgccagccattgcccatactatcgagcaagcgcgtcaaggcgacctgcacttcctcgtgttgctgttggttgcagtattagtatttgcagtgacgttctttgttgtatttgttgagcgtggtcaacgccgcattgtggtaaactacgcgaaacgtcagcaaggtcgtcgtgtctatgctgcacagagcacacatttaccgctgaaagtgaatatggcgggggtaatcccggcaatcttcgcttccagtattattctgttcccggcgaccatcgcgtcatggttcgggggcggtactggttggaactggctgacaacaatttcgctgtatttgcagcctgggcaaccgctttatgtgttactctatgcgtctgcaatcatcttcttctgtttcttctacacggcgttggttttcaacccgcgtgaaacagcagataacctgaagaagtccggtgcatttgtaccaggaattcgtccgggagagcaaacggcgaagtatatcgataaagtaatgacccgcctgaccctggttggtgcgctgtatattacctttatctgcctgatcccggagttcatgcgtgatgcaatgaaagtaccgttctacttcggtgggacctcactgcttatcgttgttgtcgtgattatggactttatggctcaagtgcaaactctgatgatgtccagtcagtatgagtctgcattgaagaaggcgaacctgaaaggctacggccgataagctgaaagtccaaattgtaatgactctagaggcatcaaataaaacgaaaggctcagtcgaaagactgggcctttcgttttatctgttgtttgtcggtgaacgctctcctgagtaggacaaatccgccgccct

SEQ ID NO: 19 - DNA, 561 bp, Artificial Sequence
  knock-in cassette
taaccctcactaaagggcggaagttcctattctctagaaagtataggaacttcagcacgtgttgacaattaatcatcggcatagtatatcggcatagtataatacgacaaggtgaggaactaaaccatggctaaactgacttctgcagttccggttctgactgctcgtgatgttgctggtgctgttgaattttggactgatcgtctgggtttttctcgtgattttgttgaagatgattttgctggtgttgttcgtgatgatgttactctgtttatttccgctgttcaggatcaggttgttccggataacactctggcttgggtttgggttcgtggtctggatgaactgtatgctgaatggtctgaagttgtttctactaactttcgtgatgcttccggtccggcaatgactgaaattggtgaacagccgtggggtcgtgaatttgcactgcgtgatccggcaggtaactgtgtacattttgtagcagaagaacaggattaatgagaagttcctattctctagaaagtataggaacttcgagccctaatgaactccgtgcta

SEQ ID NO: 20 - DNA, 69 bp, Artificial Sequence
  primer
tcgtgctgctatcgaagctgctggcggtaaaatcgaggaataagtagcagtaaccctcactaaagggcg

SEQ ID NO: 21 - DNA, 71 bp, Artificial Sequence
  primer
cggaagcacgaactttcatttttactctccgtaacttctcgggcgaccaatagcacggagttcattagggc

SEQ ID NO: 22 - DNA, 6838 bp, Artificial Sequence
  plasmid
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggctaaacaaccgggattagattttcaaagtgccaaaggtggcttaggcgagctgaaacgcagactgctgtttgttatcggtgcgctgattgtgttccgtattggctcttttattccgatccctggtattgatgccgctgtacttgccaaactgcttgagcaacagcgaggcaccatcattgagatgtttaacatgttctctggtggtgctctcagccgtgcttctatctttgctctggggatcatgccgtatatttcggcgtcgatcattatccagctgctgacggtggttcacccaacgttggcagaaattaagaaagaaggggagtctggtcgtcgtaagatcagccagtacacccgctacggtactctggtgctggcaatattccagtcgatcggtattgctaccggtctgccgaatatgcctggtatgcaaggcctggtgattaacccgggctttgcattctacttcaccgctgttgtaagtctggtcacaggaaccatgttcctgatgtggttgggcgaacagattactgaacgaggtatcggcaacggtatttcaatcattatcttcgccggtattgtcgcgggactcccgccagccattgcccatactatcgagcaagcgcgtcaaggcgacctgcacttcctcgtgttgctgttggttgcagtattagtatttgcagtgacgttctttgttgtatttgttgagcgtggtcaacgccgcattgtggtaaactacgcgaaacgtcagcaaggtcgtcgtgtctatgctgcacagagcacacatttaccgctgaaagtgaatatggcgggggtaatcccggcaatcttcgcttccagtattattctgttcccggcgaccatcgcgtcatggttcgggggcggtactggttggaactggctgacaacaatttcgctgtatttgcagcctgggcaaccgctttatgtgttactctatgcgtctgcaatcatcttcttctgtttcttctacacggcgttggttttcaacccgcgtgaaacagcagataacctgaagaagtccggtgcatttgtaccaggaattcgtccgggagagcaaacggcgaagtatatcgataaagtaatgacccgcctgaccctggttggtgcgctgtatattacctttatctgcctgatcccggagttcatgcgtgatgcaatgaaagtaccgttctacttcggtgggacctcactgcttatcgttgttgtcgtgattatggactttatggctcaagtgcaaactctgatgatgtccagtcagtatgagtctgcattgaagaaggcgaacctgaaaggctacggccgataagcggccgctagcacggagttcattagggctcgaagttcctatactttctagagaataggaacttctcattaatcctgttcttctgctacaaaatgtacacagttacctgccggatcacgcagtgcaaattcacgaccccacggctgttcaccaatttcagtcattgccggaccggaagcatcacgaaagttagtagaaacaacttcagaccattcagcatacagttcatccagaccacgaacccaaacccaagccagagtgttatccggaacaacctgatcctgaacagcggaaataaacagagtaacatcatcacgaacaacaccagcaaaatcatcttcaacaaaatcacgagaaaaacccagacgatcagtccaaaattcaacagcaccagcaacatcacgagcagtcagaaccggaactgcagaagtcagtttagccatggtttagttcctcaccttgtcgtattatactatgccgatatactatgccgatgattaattgtcaacacgtgctgaagttcctatactttctagagaataggaacttccgccctttagtgagggttaattgcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 23 - DNA, 6266 bp, Artificial Sequence
  plasmid
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggctaaacaaccgggattagattttcaaagtgccaaaggtggcttaggcgagctgaaacgcagactgctgtttgttatcggtgcgctgattgtgttccgtattggctcttttattccgatccctggtattgatgccgctgtacttgccaaactgcttgagcaacagcgaggcaccatcattgagatgtttaacatgttctctggtggtgctctcagccgtgcttctatctttgctctggggatcatgccgtatatttcggcgtcgatcattatccagctgctgacggtggttcacccaacgttggcagaaattaagaaagaaggggagtctggtcgtcgtaagatcagccagtacacccgctacggtactctggtgctggcaatattccagtcgatcggtattgctaccggtctgccgaatatgcctggtatgcaaggcctggtgattaacccgggctttgcattctacttcaccgctgttgtaagtctggtcacaggaaccatgttcctgatgtggttgggcgaacagattactgaacgaggtatcggcaacggtatttcaatcattatcttcgccggtattgtcgcgggactcccgccagccattgcccatactatcgagcaagcgcgtcaaggcgacctgcacttcctcgtgttgctgttggttgcagtattagtatttgcagtgacgttctttgttgtatttgttgagcgtggtcaacgccgcattgtggtaaactacgcgaaacgtcagcaaggtcgtcgtgtctatgctgcacagagcacacatttaccgctgaaagtgaatatggcgggggtaatcccggcaatcttcgcttccagtattattctgttcccggcgaccatcgcgtcatggttcgggggcggtactggttggaactggctgacaacaatttcgctgtatttgcagcctgggcaaccgctttatgtgttactctatgcgtctgcaatcatcttcttctgtttcttctacacggcgttggttttcaacccgcgtgaaacagcagataacctgaagaagtccggtgcatttgtaccaggaattcgtccgggagagcaaacggcgaagtatatcgataaagtaatgacccgcctgaccctggttggtgcgctgtatattacctttatctgcctgatcccggagttcatgcgtgatgcaatgaaagtaccgttctacttcggtgggacctcactgcttatcgttgttgtcgtgattatggactttatggctcaagtgcaaactctgatgatgtccagtcagtatgagtctgcattgaagaaggcgaacctgaaaggctacggccgataagcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

(19) United States
(12) Patent Application Publication
(10) Pub. No.: US 2024/0271084 A1
(43) Pub. Date: Aug. 15, 2024

(54) MICROORGANISM STRAIN AND METHOD FOR ANTIBIOTIC-FREE PLASMID-BASED FERMENTATION

(71) Applicant: GEN-H GENRTIC ENGINEERING HEIDELBERG GMBH, Heidelberg (DE)

(72) Inventors: Harald KRANZ, Leimen (DE); Marlen SCHMIDT, Heidelberg (DE); Marion FOERSCHLE, Heidelberg (DE)

(21) Appl. No.: 18/562,183

(22) PCT Filed: May 19, 2022
(86) PCT No.: PCT/EP2022/063497

§ 371 Date: Nov. 17, 2023

(30) Foreign Application Priority Data
May 20, 2021 (EP) 21174990.8

(51) Int. Cl. C12N 1/20; C12N 15/70; C12R 1/19

(52) U.S. Cl. CPC C12N 1/205; C12N 15/70; C12N 2510/02; C12R 2001/19

(57) ABSTRACT

The present invention relates to means for recombinant manufacture. In particular, it relates to a recombinant bacterial cell comprising in its genome (i) at least one endogenous essential gene which is inactivated, and (ii) at least one introduced copy of the said at least one essential gene, wherein said introduced copy is operatively linked to a heterologous expression control sequence being inducible by an inducer molecule, such that the expression of said essential gene in the recombinant bacterial cell is dependent on the presence of said inducer molecule. The present invention, further relates to a method for generating the recombinant bacterial cell, a method for recombinant manufacture of a compound of interest and the use of a recombinant bacterial cell for the manufacture of a compound of interest. Moreover, the invention provides a kit for recombinant manufacture of a compound of interest.

The present invention relates to means for recombinant manufacture. In particular, it relates to a recombinant bacterial cell comprising in its genome (i) at least one endogenous essential gene which is inactivated, and (ii) at least one introduced copy of the said at least one essential gene, wherein said introduced copy is operatively linked to a heterologous expression control sequence being inducible by an inducer molecule, such that the expression of said essential gene in the recombinant bacterial cell is dependent on the presence of said inducer molecule.

The present invention, further relates to a method for generating the recombinant bacterial cell, a method for recombinant manufacture of a compound of interest and the use of a recombinant bacterial cell for the manufacture of a compound of interest. Moreover, the invention provides a kit for recombinant manufacture of a compound of interest.

Plasmid-based expression of genes is an important tool for industrial-scale manufacture of chemical or pharmaceutical products. Typically, bacteria, such as E. coli, are transformed with an expression plasmid encoding a gene of interest required for the recombinant manufacture of a desired chemical or pharmaceutical product. Conventional cultivation methods require selection markers, such as resistance genes against antibiotics, in the expression plasmid and the use of antibiotics in the culture in order to select transformed bacteria and subsequently keep the expression plasmids stable in said transformed bacteria. However, the excessive use of antibiotics is expensive and causes various environmental and health problems, e.g., it facilitates the generation of multi-resistant pathogens.

Several techniques have been developed for the recombinant manufacture of desired products which are independent of the use of antibiotics. For example, a toxin gene was introduced into a bacterial chromosome and will be expressed. Detoxification is achieved if a plasmid is introduced which in addition to a gene of interest also expresses a detoxification gene (Szpirer 2005).

In another approach, an essential gene is downregulated by a repressor. Only if an expression plasmid is introduced that comprises a gene of interest and allows for removal of the repressor due to the presence of an operator, the bacterial can be propagated (Cranenburgh 2001). RNA-based selection markers have been developed. In such an approach, a marker on a chromosome will be neutralized by an antisense RNA expressed from the successfully transformed expression plasmid comprising the gene of interest (Mairhofer 2010).

Amino acid auxotrophy has been used in another approach. A genomic deletion of the proBA gene is complemented by proBA on the expression plasmid comprising, in addition, the gene of interest (Fiedler 2001).

A similar approach of complementation has been reported for the essential infA gene which is deleted in the genome and becomes complemented by a successfully transformed expression plasmid such that the gene of interest becomes expressed in the transformed bacteria (WO2003/000881).

Further approaches are based on bacteria that are stably transformed with helper plasmids which express the essential infA gene and comprise a temperature sensitive origin of replication (ori) and a resistance gene for a first selection marker. Those bacteria are grown at a temperature allowing the ori to operate. After transformation with an expression plasmid having a normal ori and a further selection marker, successful transformed bacteria can be selected and grown on normal temperature and in the presence of the selection agent for the selection marker of the expression plasmid (WO2017/097383). Bacteria that are treated by a heat shock, however, develop a stress response which is inferior for recombinant manufacture.

Moreover, thermo-sensitive approaches also use leader sequences for the infA. The endogenous infA expression is inhibited at a lower temperature by a leader sequence forming a hairpin structure at the 5′end of the transcript. The expression plasmid comprises in addition to the gene of interest an infA gene which can be transcribed and translated properly at lower temperatures. Accordingly, successfully transformed bacteria will grow and be viable at lower temperatures in such an approach while untransformed bacteria will die because the endogenous infA transcripts cannot be translated into infA protein (WO2018/083116). Bacteria can only be propagated at low temperatures which is inferior for recombinant manufacturing.

However, it would be highly desirable to develop production bacterial strains expressing a desired gene of interest which are independent on complex and cumbersome cloning and the aforementioned mechanisms such as temperature shifts.

The technical problem underlying the present invention may be seen as the provision of means and methods for complying with the aforementioned needs. The technical problem is solved by the embodiments characterized in the claims and herein below.

The present invention relates to a recombinant bacterial cell comprising in its genome:
(i) at least one endogenous essential gene which is inactivated, and
(ii) at least one introduced copy of the said at least one essential gene, wherein said introduced copy is operatively linked to a heterologous expression control sequence being inducible by an inducer molecule, such that the expression of said essential gene in the recombinant bacterial cell is dependent on the presence of said inducer molecule.

It is to be understood that in the specification and in the claims, “a” or “an” can mean one or more, depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” or similar terms are used in conjunction with features in order to indicate that these features are preferred but not mandatory features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “recombinant” as used herein refers to a bacterial cell which has been genetically modified. Preferably, said genetic modification may be a permanent or transient modification.

Accordingly, encompassed in accordance with the present invention are any modifications of the genome of the bacterial cell including those achieved by recombination techniques and those achieved by gene editing techniques. Moreover, encompassed are also any modifications which result from the introduction of plasmid DNA into the bacterial cell. The person skilled in the art is well aware of how modifications resulting in a recombinant bacterial cell can be carried out. Modifications of the genome may be carried out, e.g., by using CRISPR-Cas-9 systems, site-specific recombination techniques including, e.g., lambda red recombination-based systems (the technology is also called Red/ET recombination or Red/ET recombineering), Cre-recombinase-based systems, FLP-FRT-based systems, transposon-based techniques or random integration techniques. Plasmid DNA may be introduced into a bacterial cell, e.g., by using electroporation, TSS-, CaCl— or RbCl-transformation techniques or virus-mediated transduction.

The term “bacterial cell” as used herein refers to a cell of gram-positive or gram-negative bacteria suitable for the recombinant manufacture of a compound of interest.

Depending on the compound of interest to be manufactured and the amount of compound of interest envisaged, the skilled person is well aware of what bacterial cell is suitable. Typically, the bacterial cell is selected from the group consisting of Escherichia coli, Bacillus species, preferably, Bacillus subtilis, Bacillus lichenformis, Bacillus lentus, Bacillus amyloliquefaciens, Bacillus brevis, Bacillus stearothermophilus, Bacillus clausii, Bacillus alkalophilus, Bacillus coagulans, Bacillus circulans, Bacillus pumilus, Bacillus thuringiensis, Bacillus psuedofirmus, Bacillus marmarensis, Bacillus cellulolyticus, Bacillus hemicellulolyticus, Bacillus clarkia, Bacillus akibai, Bacillus gibsonii, Bacillus lautus, Bacillus megaterium or Bacillus halodurans, Lactobacillus species, preferably, Lactobacillus plantarum, Lactobacillus brevis, Lactobacillus corynformis, Lactobacillus sanfranciscensis, Lactobacillus acidophilus, Lactobacillus delbrueckii, Lactobacillus helveticus, Lactobacillus curvatus or Lactobacillus varians, and Acetobacter species, preferably, Acetobacter aceti or Acetobacter pasteurianus. Corynebacterium species, preferably, Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola, Corynebacterium effiziens, Corynebacterium efficiens, Corynebacterium deserti, Brevibacterium flavum, Brevibacterium lactofermentum, Brevibacterium divarecatum, Pseudomonas putida, Pseudomonas syringae, Streptomyces species, preferably, Streptomyces coelicolor, Streptomyces lividans, Streptomyces albus, Streptomyces avermitilis, Gluconobacter oxydans, Gluconobacter morbifer, Gluconobacter thailandicus, Clostridium spezies, preferably, Clostridium acetobutylicum, Clostridium saccharobutylicum, Clostridium beijerinckii, Streptococcus spezies, preferably, Streptococcus aureus, Streptococcus equisimilis, Streptococcus pyogenes, Streptococcus uberis, Streptococcus equi subsp., Zooepidemicus, and Basfia succiniciproducens. Preferably, the bacterial cell referred to herein is a gram-negative bacterial cell. Preferably, said bacterial cell is an E. coli cell.

The term “endogenous essential gene” as used herein refers to a gene which is naturally occurring in the genome of the bacterial cell and which is required for proper function of the bacterial cell. The absence of a functional endogenous essential gene as referred to herein shall, typically, result in lethality and/or growth arrest of the bacterial cell. Preferably, the endogenous essential gene required for proper cell growth and/or viability of said bacterial cell. More preferably, the at least one endogenous essential gene is selected from the group consisting of infA, infCJIF-3, dnaJ, dnaK, era, frr, ftsL, ftsN, ftsZ, grpE, mopA, mopB, msbA, nusG, parC, rpsB, secY and trmA. Most preferably, said at least one essential gene is infA or secY.

Nucleic acid sequence of the aforementioned essential genes are known in the art for various bacterial species including those bacterial species explicitly mentioned elsewhere herein. Besides those nucleic acid sequences disclosed in the prior art, the aforementioned endogenous essential genes may also encompass variant sequences. Preferably, such a variant sequence still encodes a gene product which is capable to exert the biological function of the gene product of the essential gene. Typically, the variant nucleic acid sequence is at least 70%, at least 75%, at least 80%, at least 85%, at least 90%, at least 95%, at least 96%, at least 97%, at least 98% or at least 99% identical to the specific sequence for the endogenous essential gene disclosed in the prior art. Yet the endogenous essential genes may also encompass those which comprise nucleic acid sequences which encode gene products, i.e., proteins having an amino acid sequences which is at least 70%, at least 75%, at least 80%, at least 85%, at least 90%, at least 95%, at least 96%, at least 97%, at least 98% or at least 99% identical to the specific sequence for the gene product encoded by endogenous essential gene disclosed in the prior art. Nucleic acid sequence identity or amino acid sequence identity as referred to herein can be, preferably, determined by determining the number of identical amino acids between two nucleic acid sequences or amino acid sequences wherein the sequences are aligned so that the highest order match is obtained. It can be calculated using published techniques or methods codified in computer programs such as, for example, BLASTP, BLASTN or FASTA. The percent sequence identity values are, typically, calculated over the entire nucleic acid or amino acid sequence or over a fragment of the nucleic acid or amino acid sequences being at least 50% in length of the entire sequences. For calculation of the percentage of sequence identity, typically, the default settings of the aforementioned computer programs are to be used. Preferred nucleic acid sequences for the open reading frames of the aforementioned essential genes from infA and secYE. coli are also shown in SEQ ID NO: 1 (infA) and SEQ ID NO: 2 (secY), respectively.

The term “inactivated” as used herein mean that the endogenous essential gene is no longer biologically active. This is typically the result of a genetic modification as referred to elsewhere herein. In particular, it is envisaged that the inactivated endogenous essential gene comprises at least one nucleotide deletion, substitution and/or addition compared to the wild type version. The result of said genetic modification may be that no gene product is produced at all or that an inactivated version of the gene product is made which is not capable of exerting the biological function properly. Preferably, the endogenous essential gene or elements governing its expression are removed from the genome of the bacterial cell. Alternatively, the coding sequence of the gene may be modified such that there will be a termination signal at the beginning. Preferably, the endogenous essential gene in the genome of the bacterial system is inactivated by using CRISPR-Cas-9 systems, site-specific recombination techniques including, e.g., lambda red recombination-based systems, Cre-recombinase-based systems, FLP-FRT-based systems, transposon-based techniques or random integration techniques. Yet, the endogenous essential gene may also be inactivated by other techniques such as random mutagenesis and selection for inactivated bacterial cells. For random mutagenesis, bacterial cells are treated with a mutagenizing agent or radiation in order to introduce randomly DNA modifications, such as substitutions or deletions. Subsequently, those bacterial cell mutant clones are identified which lack the essential gene, typically, by introducing at least one copy of the said at least one essential gene, wherein said copy is operatively linked to a heterologous expression control sequence being inducible by an inducer molecule, such that the expression of said essential gene in the mutant bacterial cell is dependent on the presence of said inducer molecule, and cultivating the mutant bacterial cells in the presence of the inducer molecule as described elsewhere herein in detail. Preferably, an inactivated endogenous essential gene as referred to in accordance with the present invention, however, is a deleted endogenous essential gene. Moreover, it is, preferably, envisaged that the bacterial genome lacks any gene being capable of functionally complementing the at least one essential gene in the absence of the inducer molecule. More preferably, inactivation may be carried out as described in the Examples, below. Preferred knock out constructs are also described in the Examples, below.

The term “introduced copy of the said at least one essential gene” as used herein refers to a copy of the at least one essential gene which has been introduced into the bacterial cell, typically, before or simultaneously to the inactivation of the at least one endogenous essential gene. The said introduced copy of the at least one essential gene is operatively linked to a heterologous expression control sequence being inducible by an inducer molecule, such that the expression of said essential gene in the recombinant bacterial cell is dependent on the presence of said inducer molecule as describe elsewhere herein in more detail. Preferably, the introduced copy of the said at least one essential gene and the heterologous expression control sequence being inducible by an inducer molecule operatively linked thereto are comprised in an expression construct that is introduced into the genome of the bacterial cell by using, e.g., random or side specific recombination techniques as described elsewhere herein. Yet, the introduced copy of the said at least one essential gene and the heterologous expression control sequence being inducible by an inducer molecule operatively linked thereto may be comprised in a plasmid which has been introduced into the bacterial cell. Typical plasmids which may be used are selected from the group consisting of. pJF118EH, pKK223-3, pUC18, pBR322, pACYC184, pASK-IBA3, pET and any expression plasmid derived therefrom. More preferably, the plasmid may be a plasmid as described in the accompanying Examples, below.

The term “operatively linked” as used herein means that two genetic elements referred to herein are functionally linked to each other. For example, an expression control sequence may be functionally linked to the coding sequence of a gene of interest if it is capable of governing its expression. Typically, the expression sequence may be positioned in physical proximity to the coding sequence of the gene, i.e. at its 5′end. However, it is also possible to position an expression control sequence at a certain distance to the coding sequence as long as it is still capable to govern the expression thereof. The skilled person is well aware of how such operatively linkage can be achieved depending on a given expression control sequence.

The term “heterologous expression control sequence being inducible by an inducer molecule” as referred to herein relates to a nucleic acid sequence which is capable when being operatively linked to a coding nucleic acid sequence of a gene to govern the expression of said coding nucleic acid sequence of the gene. The expression control sequence is typically a promoter comprising transcription and translation regulating elements. The expression control sequence shall be inducible by an inducer molecule, i.e. its function shall depend on the presence and/or amount of an inducer molecule. Such inducer-dependent expression control sequences are known in the art and encompass, preferably, AraC PBAD, RhaR-RhaS rhaBAD, XylS Pm, NitR/PnitA and ChnR/Pb inducer/promoter systems (see, e.g., Brautaset 2009, Microb Biotechnol 2(1): 15-30). Other inducer-dependent systems may be the tetracycline-inducible system (tetON/OFF), the lacI/IPTG, the ethanol-inducible system (AlcA/AlcR), the streoid-inducible system (LexA/XVE) or the vanillate-inducible system. Moreover, the inducible expression control sequence referred to herein shall be heterologous with respect to the wild type, i.e. naturally occurring, bacterial cell, i.e. it shall be an expression control sequence which does not naturally occur in the recombinant bacterial cell or which is not natively associated with the endogenous essential gene in the said bacterial cell.

The term “inducer molecule” as referred to in accordance with the present invention relates to any molecule that is capable of acting as an inducer of expression of a gene being operatively linked to the heterologous expression control sequence being inducible by said inducer molecule. Typically, inducer molecules may be metabolic product which are metabolized by bacteria such as sugars, preferably, arabinose, rhamnose, xylose or sucrose, substituted benzenes, cyclohexanone-related compounds, F-caprolactam, propionate, thiostrepton, alkanes, isopropyl β-D-1-thiogalactopyranoside, tetracycline, anhydrotetracycline or peptides. Preferably, said inducer molecule is selected from the group consisting of: arabinose, rhamnose, xylose, sucrose, tetracycline, anhydrotetracycline and IPTG.

Advantageously, it has been found in accordance with the studies underlying the present invention that by inactivating an endogenous essential gene in the genome of a bacterial cell and introducing a copy of said essential gene under the control of an expression control sequence which can be controlled by an inducer molecule, a recombinant bacterial cell can be provided in which the viability and/or growth depends on the presence or amount of the inducer molecule in the culture. Such a recombinant bacterial cell can be used as a common platform for generating recombinant bacterial cells useful for the manufacture of a variety compounds of interest, i.e. protein producing bacterial cells. Thanks to the provision of the aforementioned recombinant bacterial cell as a common platform, it is no longer necessary to generate bacterial cells producing a desired compound of interest without the use of antibiotics in a cumbersome and inefficient manner. In particular, some prior art techniques require individual generation of the producing bacterial cells from scratch, i.e., the unmodified bacterial cells, using individual complementing plasmids for each case or, in the case of knock out approaches, the application of helper plasmids in order to remove resistance genes from the final producing bacterial cells.

Thanks to the present invention a common bacterial cell can be used as a platform. A given expression plasmid used to generate a producing bacterial cell can be simply modified with less cloning efforts to comply with the system. There is no need for helper plasmids and for removing them, e.g., by using heat treatment steps above 37° C. culture temperature.

It will be understood that a producing bacterial cell for a desired compound of interest to be used in a recombinant manufacturing process can be generated from the aforementioned recombinant bacterial cell by introducing an expression plasmid into said recombinant bacterial cell.

Thus, in a preferred embodiment of the recombinant bacterial cell of the present invention, said bacterial cell comprises an expression plasmid which comprises at least one plasmid copy of the at least one essential gene, wherein said plasmid copy is under the control of the expression control sequence which is biologically active in said bacterial cell.

Preferably, said expression plasmid is selected from the group consisting of pJF118EH, pKK223-3, pUC18, pBR322, pACYC184, pASK-IBA3, pET and any expression plasmid derived therefrom. It will be understood that although some plasmids may comprise selection markers such as antibiotic resistance genes, those genes are not required for using the recombinant bacterial cell according to the invention. Rather, the expression of the plasmid copy of the at least one essential gene shall be sufficient for selecting those recombinant bacterial cells which have been successfully taken up the expression plasmid. More preferably, the expression plasmid may be a plasmid as described in the accompanying Examples, below.

It is envisaged in accordance with the present invention that the expression of the plasmid copy of the at least one essential gene is governed by an expression control sequence which is biological active under physiological conditions in the recombinant bacterial cell. Thus, said expression control sequence may be a constitutively active promoter which is permanently expressed in the cell. Alternatively, it may be a promoter which is constitutively expressed during the phase of growth in the recombinant bacterial cell. Suitable expression control sequences are well known to the person skilled in the art. They include, e.g., the constitutively active promoters from E. coli described in Shimada et al. 2014, PLoS ONE 9(3): e90447. https://doi.org/10.1371/journal.pone.0090447. Similar constitutively active promoters can be found for other bacteria and have been reported in the prior art already. Preferably, said expression control sequence which is biologically active is the expression control sequence which is natively associated with the at least one endogenous essential gene. Thus, more preferably, the said expression control sequence is a promoter selected from the group consisting of infA promoter, infAp2 promoter, infC IF-3 promoter, dnaJ promoter, dnaK promoter, era promoter, frr promoter, ftsL promoter, ftsN promoter, ftsZ promoter, grpE promoter, mopA promoter, mopB promoter, msbA promoter, nusG promoter, parC promoter, rpsB promoter, secY promoter and trmA promoter. Most preferably, said at least one essential gene is infA promoter.

Preferably, said expression plasmid comprises at least one further nucleic acid of interest which is required for the recombinant manufacture of a compound of interest in said recombinant bacterial cell.

The term “at least one further nucleic acid of interest” as used herein relates to a nucleic acid which encodes a gene product, i.e. a protein, peptide or RNA, which is required to manufacture a compound of interest envisaged to be produced by the recombinant bacterial cell of the invention. It will be understood that the compound of interest to be manufacture may also be the encoded gene product itself. Typically, however, the gene product shall be required for the recombinant manufacture of the compound of interest. Preferably, a protein as referred to herein may be an enzyme which is required for one or more catalytic conversions required during the recombinant manufacturing process. Alternatively, it may be a binding protein, such as a chaperone, which binds to and protects a compound of interest and, thus, may facilitate purification of said compound. A peptide referred to herein may be useful for growth control of the bacterial cells within the culture or may be useful as antimicrobial peptides if the peptide itself is envisaged to be produced as a compound of interest. An RNA as referred to herein may be an inhibitory RNA, such as siRNA or microRNA, or an enzymatically active RNA. Inhibitory RNAs or ribozymes are both suitable agents for controlling the presence and or amount of certain other RNAs such as mRNAs encoding certain proteins or peptides. By controlling such mRNAs, the inhibitory RNAs or ribozymes may control the presence or amount of biological activity of a protein present in the bacterial cell, e.g., the presence or amount of enzymatic activity. It will be understood that a recombinant manufacturing process can, hence, be controlled by those agents as well.

It will be understood that the nucleic acid of interest required for the recombinant manufacture of the compound of interest shall be operatively linked to an expression control sequence which is biologically active in the bacterial cell of the present invention or which can be activated during culture. Accordingly, such an expression control sequence may comprise a constitutively active promoter as well as an inducible promoter. Constitutively active promoters have described elsewhere herein already. It will be understood that an inducible promoter used for governing expression of the nucleic acid of interest shall not be controlled by the inducible molecule used for controlling the expression of the at least one introduced copy of the said at least one essential gene according to the present invention. Moreover, it is to be understood that if an inducible promoter is used for producing the compound of interest, the said promoter must be induced, i.e. the inducing stimulus must be applied to the recombinant bacterial cell culture. Typical inducing stimuli applied in this context may be altered physical conditions, e.g., the application of a heat stimulus (also called “heat shock”) or the administration of an inducing molecule. Inducible promoters, typically, encompass heat-inducible promoters as well as compound inducible promoters such as those referred to elsewhere herein. The skilled artisan is well aware of which promoters may be used in this context.

Care should be taken that the expression plasmid, in principle, allows for expression of both, the at least one plasmid copy of the at least one essential gene as referred to above and the at least one further nucleic acid of interest as specified above. Thus, both expression cassettes need to be arranged in the expression plasmid such that there is no interference between the expression of either expression cassette by the other one.

The term “compound of interest” as used in accordance with the present invention refers to any compound which can be recombinantly manufactured in the recombinant bacterial cell of the present invention. The compound of interest shall be, typically, manufactured due to the expression of the gene product encoded by the further nucleic acid of the expression plasmid in the producing bacterial cells described herein before. Preferably, the compound of interest belongs into a chemical class selected from the group consisting of. lipids, amino acids, purins including nucleotides and nucleosides based thereon, pyrimidines including nucleotides and nucleosides based thereon, isoprenoids, proteins, peptides, nucleic acids, terpenes, carotenoids, saccharides, polysaccharides, alkaloids, alcohols, antibiotics, vitamins, polyhydroxyalkanoates, polyamides, polylactic acids, coenzymes, organic acids, and the like. Yet, the compound of interest may also be the gene product itself encoded by the further nucleic acid of interest comprised in the expression plasmid. Typically, the compound of interest in such a case is a protein, peptide or RNA molecule. Moreover, the expression plasmid itself or a part thereof, i.e. a DNA molecule, may be envisaged as a compound of interest manufactured by the producing recombinant bacterial cell of the present invention.

Advantageously, it has been further found in accordance with the studies underlying the present invention that using a recombinant bacterial cell having inactivated an endogenous essential gene in its genome and introduced a copy of said essential gene under the control of an expression control sequence which can be controlled by an inducer molecule as a platform, various different producing bacterial cells can be easily generated that can be applied for the recombinant manufacture of a desired compound of interest. Said platform bacterial cells can be cultivated in the presence of the inducer molecule. After transformation with an expression plasmid as described herein, the transformed bacterial cells will no longer be cultured in the presence of the inducer molecule and only those bacteria will grow and/or be viable which have taken up the expression plasmid which allows for expression of the at least one essential gene.

There is no antibiotic selection marker and no cultivation under selective conditions necessary since selection of the transformed bacteria takes place under physiological conditions once the inducer molecule is withdrawn from the culture. Thus, the recombinant bacterial cells of the present invention avoid the use of antibiotics which are required in conventional recombinant manufacturing process for selection and cultivation. Neither the expression plasmid which is introduced nor the platform bacterial cells require antibiotics during culture or selection and culture. The expression plasmid in a producing bacterial cell is kept stable in the cell even under high density fermentation conditions and under optimal and suboptimal culture temperatures and conditions.

The present invention also relates to a method for generating the recombinant bacterial cell of the present invention comprising the steps of:
(a) introducing into the genome of a bacterial cell at least one copy of at least one essential gene being operatively linked to a heterologous expression control sequence being inducible by an inducer molecule; and
(b) inactivating the at least one endogenous essential gene in the genome of the bacterial cell, such that the expression of said essential gene in the recombinant bacterial cell is dependent on the presence of inducer molecule.

The method according to the present invention may consist of the aforementioned steps (a) and (b) or may comprise further steps, such as cultivating and/or pretreating the bacterial cell prior to step (a) and/or cultivating and/or treating the bacterial cell after step (b).

It will be understood that the viability and/or growth of the recombinant bacterial cell after having carried out the aforementioned method of the present invention will become dependent on the expression of the at least one introduced copy of the at least one essential gene. Said expression, however, is dependent on the presence and/or amount of an inducer molecule in the culture. Accordingly, the bacterial cell is, preferably, cultivated after step (b) in the presence of the inducer molecule, preferably, an inducer molecule as specified elsewhere herein.

By cultivating the recombinant bacterial cell in the presence of the inducer molecule, cell cultures of the recombinant bacterial cells can be generated which can be used as a common platform for the manufacture of various compounds of interest. Depending on the desired compound of interest, the recombinant bacterial cells of the said cultures may be individually transformed with an expression plasmid comprising at least one plasmid copy of the at least one essential gene, wherein said plasmid copy is under the control of the expression control sequence which is biologically active in said bacterial cell and wherein said expression plasmid comprises a nucleic acid of interest which is required for the manufacture of the compound of interest.

In step (a) of the aforementioned method of the present invention, at least one copy of at least one essential gene being operatively linked to a heterologous expression control sequence being inducible by an inducer molecule is introduced into the genome of a bacterial cell as specified elsewhere herein as a host. The said at least one copy of the at least one essential gene being operatively linked to a heterologous expression control sequence is typically integrated into the genome of the bacterial cell by using site-specific or random recombination techniques as described elsewhere herein in more detail and, in particular, lambda red recombination (also called Red/ET recombination or Red/ET recombineering). How to carry out such recombination techniques is well known to the skilled artisan.

After the at least one copy of at least one essential gene being operatively linked to a heterologous expression control sequence being inducible by an inducer molecule has been introduced into the genome of the bacterial cell, step (b) of the method shall be carried out. In step (b), the at least one endogenous copy of the at least one essential gene present in the bacterial genome shall be inactivated. This inactivation can be done by various techniques. As a result of the inactivation, the gene product encoded by the at least one essential gene shall no longer be biologically active. Thus, inactivation may be achieved by removing the entire essential gene or parts thereof or it may be achieved by gene editing (e.g. using CRISPR/Cas-9 techniques) resulting in alleles of the essential gene that cannot be transcribed into RNA or the transcripts of which cannot be translated into protein. Typically, the alleles of the at least one endogenous essential gene are removed by using lambda red recombination. Any nucleic acid sequences such as selection marker sequences which may reside in the genome as a result of the lambda red recombination process may be excised using FLP or Cre recombinase based techniques. It will be understood that such selection markers need to be flanked by FRT or loxP sites.

As a result of the inactivation, the expression of said essential gene in the recombinant bacterial cell is dependent on the presence of the inducer molecule. A particular preferred technique is described in the Examples, below.

Thus, the present invention further relates to a method for recombinant manufacture of a compound of interest comprising the steps of:
(a) introducing into a recombinant bacterial cell of the present invention an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.

Preferably, said expression control sequence which is biologically active is the expression control sequence which is natively associated with the at least one endogenous essential gene. Details are to be found elsewhere herein.

Preferably, said expression plasmid comprises at least one further nucleic acid of interest which is required for the recombinant manufacture of a compound of interest in said recombinant bacterial cell.

In step (a) of the aforementioned method, an expression plasmid which comprises at least one plasmid copy of the at least one essential gene, wherein said plasmid copy is under the control of the expression control sequence which is biologically active in said bacterial cell is introduced into the said cell. Typically, the expression plasmid is to be introduced by any conventional transformation method such as electroporation, TSS-, CaCl— or RbCl-transformation techniques or virus-mediated transduction. The skilled artisan is well aware of how such transformation techniques may be carried out.

Once the expression plasmid has been introduced, e.g., by any of the aforementioned transformation techniques, the recombinant bacterial cells are cultivated in step (b) of the aforementioned method of the invention in a suitable culture medium and under suitable culture conditions. In particular, the culture medium shall be free of the inducer molecule in order to achieve selection of those recombinant bacterial cells which have successfully taken up the expression plasmid. Moreover, the culture conditions shall, typically, allow for the recombinant manufacture of the compound of interest by the cultivated recombinant bacterial cells. As described elsewhere herein, this may require applying an expression stimulus to said cultured cells. However, the compound of interest may also be constitutively manufactured in the recombinant bacterial cells. Preferably, the cultivation is done until a desired level of compound of interest is produced by the recombinant bacterial cells in the culture and the said cells and afterwards, the cells and/or the culture medium used for cultivating the cells are harvested for obtaining the compound of interest. A particular preferred technique is described in the Examples, below.

The cultivation of the recombinant bacterial cells may also comprise several cultivation steps. For example, cultivation may comprise cultivating the recombinant bacterial cell in a pre-culture until the cells are exponentially growing and subsequently inoculating a culture in a production vial such as a bioreactor with said pre-culture in order to avoid that the cells in the bioreactor culture are in a lag stage or to at least reduce said lag phase in the bioreactor.

The culture medium as referred to herein is a solution which comprises compounds that are required for proper growth and viability of the recombinant bacterial cells of the present invention. Typically, a culture medium as referred to herein comprises compounds which serve as a nitrogen and/or carbon source for the bacterial cells. Said compounds may be chemically defined compounds mixed together or obtained as a chemically undefined mixture for biological sources. Chemically defined compounds useful for culture media may be carbohydrates, organic acids, hydrocarbons, alcohols or mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Chemically undefined mixtures of compounds may be extracts from microbial, animal or plant cells, e.g., plant protein preparations, soy meal, corn meal, pea meal, corn gluten, cotton meal, peanut meal, potato meal, meat, casein, gelatins, whey, fish meal, yeast protein, yeast extract, tryptone, peptone, bacto-tryptone, bacto-peptone, wastes from the processing of microbial cells, plants, meat or animal bodies, and combinations thereof. Depending on the recombinant bacterial cells to be used, i.e. the platform recombinant bacterial cells whose growth and/or viability is dependent on the presence or certain amount of an inducer molecule in the culture medium or the producing bacterial cells obtained after transformation with an expression plasmid as specified herein whose growth and/or viability in the absence of the inducer molecule in the culture medium depend on proper uptake of the expression plasmid, a culture medium to be used in accordance with the present invention may or may not comprise further ingredients such as an inducer molecule.

A bioreactor as referred to herein a vessel in which the cultivation of cells in large scale and the large-scale production of a product of interest takes place. After termination of the cultivation in the production bioreactor the fermentation broth is harvested and the product of interest is recovered. The bioreactor may contain inlets and outlets, for example for media, and different sensors, e.g. for measuring pH and temperature during the fermentation process. The fermentation medium in the production bioreactor may be the same as or different from the fermentation medium used in the seed fermenter or the last seed fermenter in a seed train. The production bioreactor may have a volume of 500 L, 1,000 L, 5,000 L, 10,000 L, 20,000 L, 50,000 L or 100,000 L.

The cultivation as referred to before may be carried out in a batch mode wherein the cells are cultured in the initially present culture medium without any change in medium composition or the volume of the medium. Thus, in batch mode no substantial or significant amount of fresh liquid culture medium is added to the cell culture and no substantial or significant amount of liquid culture medium is removed from the cell culture during culturing. Alternatively, the culture may be carried out in a fed-batch mode wherein the cells are cultured in the initially present culture medium and a feed solution is added in a periodic or continuous manner without substantial or significant removal of liquid culture medium during culturing. Fed-batch cultures can include various feeding regimens and times, for example, daily feeding, feeding more than once per day, or feeding less than once per day, and so on. Yet, as an alternative, the culture may be carried out in a continuous fermentation mode wherein the cells are cultured in the initially present culture medium and new culture medium is continuously fed to the bioreactor and fermented culture medium is removed from the bioreactor at the same rate so that the volume in the bioreactor is constant.

Thus, in a preferred embodiment of the aforementioned method of the invention, said method further comprises:
(c) obtaining the compound of interest manufactured by the recombinant bacterial cell.

After the aforementioned method of the present invention has been performed, the compound of interest may be obtained by purification from the culture medium used for culture or from the cultivated recombinant bacterial cell(s). Typically, the fermentation broth and the bacterial cells are separated from each other, e.g., by centrifugation, sedimentation or filtration techniques.

If the compound of interest is present in the culture medium, further purification steps may depend on the chemical nature and properties of the compound of interest. Such purification steps may encompass affinity chromatography (e.g., ion exchange-, affinity-, hydrophobic-, chromatofocusing-, and size exclusion-chromatography), extraction, electrophoresis (e.g., preparative isoelectric focusing), filtration, evaporation, spray-drying, precipitation (e.g., using ammonium sulfate) or crystallization.

If the compound of interest is comprised in the recombinant bacterial, release of the compound of interest from said cells might be needed. Release from the cells can be achieved, for instance, by cell lysis with techniques well known to the skilled artisan, e.g., lysozyme treatment, ultrasonic treatment, French press or combinations thereof. Subsequently, the compound of interest released from the recombinant bacterial cells may be further purified by purification steps as referred to above.

The method of the present invention may also encompass further steps for formulating the compound of interest in a desired form. This may include the generation of granulates, tablets, powders, solutions, gels, gaseous formulations, any 3D-items, nanoparticles, coatings and the like from the compound of interest.

Depending on the nature of the compound of interest, the formulation of the compound of interest may be carried out under particular standard conditions, such as GMP.

The present invention contemplates the use of the recombinant bacterial cell of the present invention for the recombinant manufacture of a compound of interest.

Yet, the present invention relates to a kit for recombinant manufacture of a compound of interest comprising:
(i) a recombinant bacterial cell of the present invention, and
(ii) an expression plasmid which comprises at least one plasmid copy of the at least one essential gene, wherein said plasmid copy is under the control of the expression control sequence which is biologically active in said bacterial cell.

The term “kit” as used herein refers to a collection of components required for carrying out the method of the present invention for recombinant manufacture of a compound of interest. The kit shall include a recombinant bacterial cell of the present invention and an expression plasmid which comprises at least one plasmid copy of the at least one essential gene, wherein said plasmid copy is under the control of the expression control sequence which is biologically active in said bacterial cell. Preferably, said expression control sequence which is biologically active is the expression control sequence which is natively associated with the at least one endogenous essential gene. Also preferably, said expression plasmid comprises at least one further nucleic acid of interest which is required for the recombinant manufacture of a compound of interest in said recombinant bacterial cell which is required for the recombinant manufacture of a compound of interest in said recombinant bacterial cell.

Typically, the components of the kit are provided in separate containers or within a single container. The container also typically comprises instructions for carrying out the method of the present invention for recombinant manufacture of a compound of interest. Moreover, the kit may, preferably, comprise further components which are necessary for carrying out the method of the invention such as cultivation media, washing solutions, solvents, and/or reagents or means required for purification of the compound of interest.

The following embodiments are particular preferred embodiments envisaged in accordance with the present invention. All definitions an explanations of the terms made above apply mutatis mutandis.

Embodiment 1. A recombinant bacterial cell comprising in its genome
(i) at least one endogenous essential gene which is inactivated, and
(ii) at least one introduced copy of the said at least one essential gene, wherein said introduced copy is operatively linked to a heterologous expression control sequence being inducible by an inducer molecule, such that the expression of said essential gene in the recombinant bacterial cell is dependent on the presence of said inducer molecule.

Embodiment 2. The recombinant bacterial cell of embodiment 1, wherein said bacterial cell is an E. coli cell.

Embodiment 3. The recombinant bacterial cell of embodiment 2, wherein said at least one essential gene is a gene required for cell growth and/or viability, preferably, selected from the group consisting of. infA, infCJIF-3, dnaJ, dnaK, era, frr, ftsL, ftsN, ftsZ, grpE, mopA, mopB, msbA, nusG, parC, rpsB, secY and trmA.

Embodiment 4. The recombinant bacterial cell of embodiment 3, wherein said at least one essential gene is infA or secY.

Embodiment 5. The recombinant bacterial cell of any one of embodiments 1 to 4, wherein the bacterial genome lacks any gene being capable of functionally complementing the at least one essential gene in the absence of the inducer molecule.

Embodiment 6. The recombinant bacterial cell of any one of embodiments 1 to 5, wherein said bacterial cell comprises an expression plasmid which comprises at least one plasmid copy of the at least one essential gene, wherein said plasmid copy is under the control of the expression control sequence which is biologically active in said bacterial cell.

Embodiment 7. The recombinant bacterial cell of embodiment 6, wherein said expression control sequence which is biologically active is the expression control sequence which is natively associated with the at least one endogenous essential gene.

Embodiment 8. The recombinant bacterial cell of embodiment 6 or 7, wherein said expression plasmid comprises at least one further nucleic acid of interest which is required for the recombinant manufacture of a compound of interest in said recombinant bacterial cell.

Embodiment 9. The recombinant bacterial cell of any one of embodiments 1 to 8, wherein said inducer molecule is selected from the group consisting of arabinose, rhamnose, xylose, sucrose, tetracycline, anhydrotetracycline and IPTG.

Embodiment 10. A method for generating the recombinant bacterial cell of any one of embodiments 1 to 9 comprising the steps of:
(a) introducing into the genome of a bacterial cell at least one copy of at least one essential gene being operatively linked to a heterologous expression control sequence being inducible by an inducer molecule; and
(b) inactivating the at least one endogenous essential gene in the genome of the bacterial cell, such that the expression of said essential gene in the recombinant bacterial cell is dependent on the presence of inducer molecule.

Embodiment 11. The method of embodiment 10, wherein the bacterial cell is cultivated after step (b) in the presence of the inducer molecule, preferably, an inducer molecule as specified in embodiment 9.

Embodiment 12. A method for recombinant manufacture of a compound of interest comprising the steps of:
(a) introducing into a recombinant bacterial cell of any one of embodiments 1 to 5 an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.

Embodiment 13. The method of embodiment 12, wherein said expression plasmid comprises at least one further nucleic acid of interest which is required for the recombinant manufacture of a compound of interest in said recombinant bacterial cell.

Embodiment 14. The method of embodiment 12 or 13, wherein said method further comprises:
(c) obtaining the compound of interest manufactured by the recombinant bacterial cell.

Embodiment 15. The method of any one of embodiments 12 to 14, wherein said expression control sequence which is biologically active is the expression control sequence which is natively associated with the at least one endogenous essential gene.

Embodiment 16. Use of the recombinant bacterial cell of any one of embodiments 6 to 8 for the recombinant manufacture of a compound of interest.

Embodiment 17. A kit for recombinant manufacture of a compound of interest comprising:
(i) a recombinant bacterial cell of any one of embodiments 1 to 5, and
(ii) an expression plasmid which comprises at least one plasmid copy of the at least one essential gene, wherein said plasmid copy is under the control of the expression control sequence which is biologically active in said bacterial cell.

Embodiment 18. The kit of embodiment 17, wherein said expression control sequence which is biologically active is the expression control sequence which is natively associated with the at least one endogenous essential gene.

Embodiment 19. The kit of embodiment 17 or 18, wherein said expression plasmid comprises at least one further nucleic acid of interest which is required for the recombinant manufacture of a compound of interest in said recombinant bacterial cell which is required for the recombinant manufacture of a compound of interest in said recombinant bacterial cell.

All references cited in this specification are herewith incorporated by reference with respect to their entire disclosure content and the disclosure content specifically mentioned in this specification.

### FIGURES

FIG. 1: Growth control of the different strains on LB agar plates. A) Position of the four strains analysed on the plate. B) LB agar plate without addition of arabinose or antibiotics. C) LB agar plate conditioned with 0.4% L-arabinose. D) LB agar plate conditioned with 15 μg/ml chloramphenicol.

FIG. 2: Restriction map of plasmid “pQE-T7 TNFα” (SEQ ID NO: 12).

FIG. 3: Restriction map of plasmid “pQE-T7 TNFα cmR-infA” (SEQ ID NO: 16).

FIG. 4: Restriction map of plasmid “pQE-T7 TNFα infA” (SEQ ID NO: 17).

FIG. 5: Restriction digestion of four clones obtained after transformation of the platform strain with plasmid “pQE-TNFα infA”. The plasmid DNA was digested with MluI+Pad. The expected restriction fragments of 2078 bp and 3211 bp were obtained for all four clones analysed.

FIG. 6: Growth control of clones (platform strain harbouring plasmid “pQE-T7 TNFα cmR-infA”) propagated for >120 generations without selection for cm-resistance. 48 clones were streaked out on an LB agar plate (left) and on an LB agar plate conditioned with 15 μg/ml chloramphenicol to confirm the presence of plasmid “pQE-T7 TNFα cmR-infA” after seven days of passages without the addition of antibiotics. The upper four rows show platform strain “HS996 ΔinfA:FRT ΔrecAX::FRT-araC-araBp-infA” and the lower four rows show platform strain “T7E2 ΔinfA::FRT ΔrecAX::FRT-araC-araBp-infA”.

FIG. 7: Protein expressing using the antibiotic-free platform strain. A) The platform strain E. coli “T7E2 ΔinfA::FRT ΔrecAX::FRT-araC-araBp-infA” was used to express TNFα from the vector “pQE-T7 TNFα infA” in antibiotic-free LB medium. B) E. coli T7E2 was used to express TNFα from the vector “pQE-T7 TNFα cmR” in LB medium with chloramphenicol selection pressure. The gels show samples taken prior to induction (t₀) as well as at 1, 2, 3, 6, 9, 24 and 48 hours post-induction (t₁-t₄₈). Expression of TNFα (18.8 kDa) is evident in all samples post induction.

FIG. 8: Assessing plasmid maintenance of “pQE-T7 TNFα infA” by the platform strain after 48 hours of fermentation. A streak plate was made from the fermentation culture and ten isolated colonies were analysed via restriction digest with MIuI+Pad. All ten clones showed the expected banding pattern (2078 bp and 3211 bp).

FIG. 9: Restriction map of plasmid “pQE-T7 TNFα infAp2-secY-zeoR” (SEQ ID NO: 22)

FIG. 10: Restriction map of plasmid “pQE-T7 TNFα infAp2-secY” (SEQ ID NO: 23)

FIG. 11: Assessing plasmid maintenance of “pQE-T7 TNFα infAp2-secY” by the platform strain “HS996 ΔsecY::FRT ΔrecAX::cmR-araC-araBp-secY”. Plasmid DNA was isolated from single colonies after transformation of the plasmid into the platform strain. Antibiotics were neither used to select for cells which obtained the plasmid during electroporation nor to maintain the plasmid in overnight cultures. Plasmid DNA analysed via restriction digest with PvuI. All seven clones showed the expected banding pattern (1755 bp and 4511 bp).

### EXAMPLES

The following Examples shall merely illustrate the invention. They shall not be construed, whatsoever, to limit the scope thereof.

### Example 1: Preparation of a Platform Strain with an Arabinose-Inducible infA Gene

The SEQ ID NOs referred to in the following show:
SEQ ID NO: 1: Escherichia coli infA gene: 219 bp
SEQ ID NO: 2: Escherichia coli secY gene: 1332 bp
SEQ ID NO: 3: PCR Primer “KI-primer up”: 72 bp
SEQ ID NO: 4: PCR Primer “KI-primer down”: 70 bp
SEQ ID NO: 5: “FRT-ampR-FRT-araC-araBp-infA” knock-in cassette: 3048 bp
SEQ ID NO: 6: PCR Primer “cm-primer up”: 68 bp
SEQ ID NO: 7: PCR Primer “cm-primer down”: 73 bp
SEQ ID NO: 8: “FRT-cmR” knock-in cassette: 1034 bp
SEQ ID NO: 9: PCR Primer “km-primer up”: 69 bp
SEQ ID NO: 10: PCR Primer “km-primer down”: 71 bp
SEQ ID NO: 11: “kmR-FRT” knock-in cassette: 1577 bp
SEQ ID NO: 12: Plasmid “pQE-T7 TNF alpha”: 5746 bp
SEQ ID NO: 13: PCR Primer “infA-primer up”: 68 bp
SEQ ID NO: 14: PCR Primer “infA-primer down”: 77 bp
SEQ ID NO: 15: “infA-cmR” expression cassette: 1327 bp
SEQ ID NO: 16: Plasmid “pQE-T7 TNFα cmR-infA”: 6036 bp
SEQ ID NO: 17: Plasmid “pQE-T7 TNFα infA”: 5289 bp
SEQ ID NO: 18: “cmR-araC-araBp-secY” knock-in cassette: 3746 bp
SEQ ID NO: 19: “FRT-zeoR-FRT” knock-in cassette: 561 bp
SEQ ID NO: 20: PCR Primer “secY-primer up”: 69 bp
SEQ ID NO 21: PCR Primer “secY-primer down”: 71 bp
SEQ ID NO: 22: Plasmid “pQE-T7 TNFα infAp2-secY-zeoR”: 6838 bp
SEQ ID NO: 23: Plasmid “pQE-T7 TNFα infAp2-secY”: 6266 bp
   a) Insertion of a “FRT-ampR-FRT-araC-araBp-infA” cassette into the recAX genomic locus to obtain an arabinose-inducible copy of the infA gene

A DNA fragment of about 3.0 kb in size (consisting of an FRT-flanked ampR marker gene, the araC gene together with the araB promoter, and the infA gene) was amplified using the primers KI-primer up (SEQ ID NO: 3) and KI-primer down (SEQ ID NO: 4). Fifty base pair long homology arms for the recAX genomic locus were added to the fragment during the PCR reaction. The obtained PCR product (SEQ ID NO: 5) was inserted into the recAX genomic locus of E. coli strain T7E2, a derivative of BL21(DE3), or into the corresponding locus of strain HS996 by Red/ET recombination (Zhang, Y. et al. 2000; Murphy, K. C., Campellone, K. G. and Poteete, A. R. 2000). The recombineering reaction was performed as described in the manual of the “Quick & Easy E. coli Gene Deletion Kit” (Gene Bridges, Cat. No. K006). Selection for cells with chromosomal integration of the cassette was performed on LB agar plates containing 50 μg/ml ampicillin. The correctly performed knock-in was confirmed by PCR across the modified recAX locus and subsequent sequencing of the amplicon.
b) Insertion of a “FRT-cmR” cassette upstream of the infA gene in the endogenous locus

A DNA fragment consisting of a chloramphenicol resistance marker gene with a single FRT site at its 5′ end was amplified using the primers cm-primer up (SEQ ID NO: 6) and cm-primer down (SEQ ID NO: 7). Fifty base pair long homology arms for a region upstream of the infA genomic locus were added to the fragment during the PCR reaction. The obtained PCR product (SEQ ID NO: 8) was inserted between the clpA gene and the infA gene by Red/ET recombination. Selection for cells with chromosomal integration of the cassette was performed on LB agar plates containing 50 μg/ml ampicillin and 15 μg/ml chloramphenicol. The correctly performed knock-in of the cmR-marker cassette was confirmed by PCR and subsequent sequencing of the amplicon.
c) Insertion of a “kmR-FRT” cassette downstream of the infA gene in the endogenous locus

A DNA fragment consisting of a kanamycin resistance marker gene with a single FRT site at its 3′ end was amplified using the primers km-primer up (SEQ ID NO: 9) and km-primer down (SEQ ID NO: 10). Fifty base pair long homology arms for a region downstream of the infA genomic locus were added to the fragment during the PCR reaction. The obtained PCR product (SEQ ID NO: 11) was inserted between the infA and the aat gene by Red/ET recombination. Selection for cells with chromosomal integration of the cassette was performed on LB agar plates containing 50 μg/ml ampicillin, 15 μg/ml chloramphenicol and 15 μg/ml kanamycin. The correctly performed knock-in of the kmR-marker cassette was confirmed by PCR and subsequent sequencing of the amplicon.
d) FLP recombination step to remove the cmR and kmR selection marker together with the endogenous infA gene, as well as the ampR marker in the recAX locus

The three antibiotic resistance marker genes were removed by a single FLP recombination step. Cells harbouring the chromosomal modifications were transformed with plasmid 707-FLPe (Gene Bridges, Cat. No. A104). The FLP recombination step was performed following the instructions of the manufacturer with the exception that 0.4% L-arabinose was added to the medium in all FLP-recombination steps to induce expression of the genomic copy of infA.

After performing the FLP step, cells were streaked out on an LB agar plate without antibiotics but with the addition of 0.4% L-arabinose, and incubated at 37° C. overnight. The next day, single colonies were streaked out on four LB agar plates, each conditioned with 0.4% L-arabinose and either 50 μg/ml ampicillin, 15 μg/ml kanamycin, 15 μg/ml chloramphenicol or 3 μg/ml tetracycline. Colonies sensitive for all four antibiotics were further analysed.

The loss of the ampR gene in the recAX genomic locus and the loss of the cmR-infA-kmR fragment in the endogenous infA locus was confirmed by PCR and sequencing across the single FRT sites.

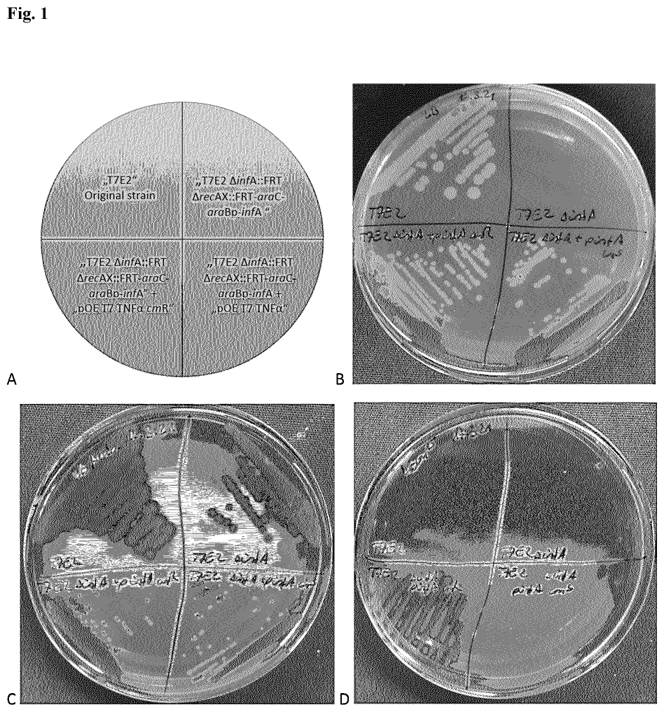

Cells were streaked out on LB agar plates with and without the addition of 0.4% L-arabinose to confirm that the obtained platform strain grows only in the presence of L-arabinose (FIG. 1). Two platform strains based on infA were prepared as described above using two E. coli strain backgrounds. E. coli strain “T7E2 ΔinfA::FRT ΔrecAX::FRT-araC-araBp-infA” is based on the strain T7E2, which is a derivative of BL21(DE3) lacking approximately 89% of the DE3 prophage. E. coli strain “HS996 ΔinfA::FRT ΔrecAX::FRT-araC-araBp-infA” is based on the E. coli K12-strain HS996. The platform strains do not carry any antibiotic resistance marker genes in the genome.

### Example 2: Preparation of the Expression Plasmids

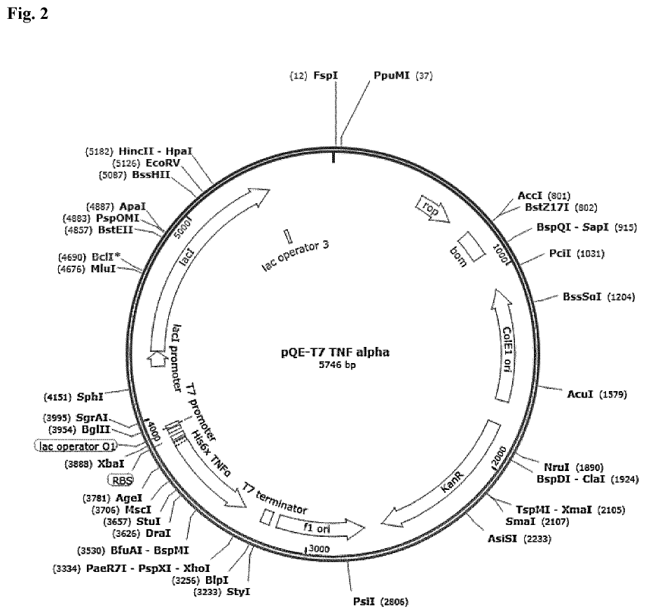

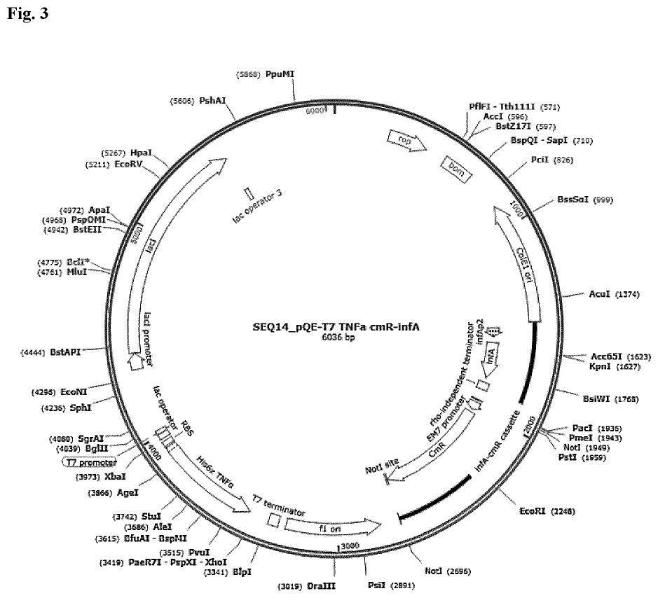

a) Replacement of the kmR selection marker gene in plasmid “pQE-T7 TNFα” by a cmR-infA cassette to obtain expression plasmid “nQE-T7 TNFα cmR-infA” (plasmid #) Plasmid “pQE-T7 TNFα” (FIG. 2, SEQ ID NO: 12) was chosen to test whether a plasmid encoding the infA gene will be maintained in the platform strain without antibiotic selection. A DNA fragment consisting of a chloramphenicol resistance marker gene flanked by NotI sites was linked to a genomic infA fragment and subsequently amplified using primers infA-primer up (SEQ ID NO: 13) and infA-primer down (SEQ ID NO: 14). The obtained PCR product (SEQ ID NO:15) was recombined with plasmid “pQE-T7 TNFα” using the recombineering proficient E. coli strain GB08-red (Fu et al. 2010). The plasmid modification was performed as described in the publication of Fu and co-workers. The fifty base pair long homology arms for the recombination with plasmid “pQE-T7 TNFα” were chosen to replace the kmR marker in the original plasmid by the “cmR-infA” cassette. The obtained plasmid “pQE-T7 TNFα cmR-infA” (FIG. 3, SEQ ID NO: 16) was used as a first test plasmid for the subsequent plasmid maintenance experiments.
b) NotI Restriction digestion of expression plasmid #I and recircularization of the plasmid without the cmR gene to obtain the expression plasmid “pQE-T7 TNFα infA” (plasmid #2)

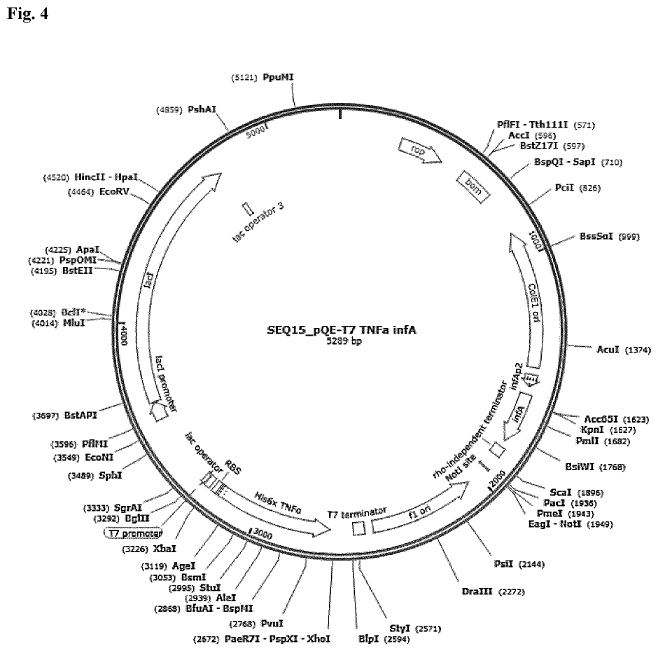

An aliquot of 100 ng plasmid DNA from “pQE-T7 TNFα cmR-infA” was digested with the restriction enzyme NotI and subsequently religated to obtain a test plasmid without an antibiotic resistance marker gene. This second test plasmid was named “pQE-T7 TNFα infA” (FIG. 4, SEQ ID NO: 17).
c) Transformation of the platform strain with the two test plasmids and growth of the cells without the addition of antibiotics or L-arabinose.

Electrocompetent cells were prepared from the platform strain as described in the manual of the “Quick & Easy E. coli Gene Deletion Kit” (Gene Bridges, Cat. No. K006). Approximately 10 ng of the two religated plasmids were each electroporated into each of the platform strains. The cells were resuspended after electroporation in 1 ml of LB medium (without antibiotics or L-arabinose) and incubated for two hours at 37° C. with shaking at 800 rpm. After this recovery phase, the cells were streaked out on LB agar plates (without antibiotics or L-arabinose) and incubated overnight at 37° C. More than one hundred colonies were visible on the plates the next day.

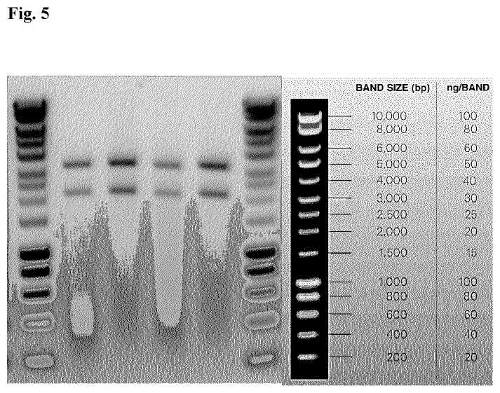

Plasmid DNA was isolated from four clones from the “pQE-T7 TNFα infA” transformation to confirm the presence of the plasmid, even though the plasmid does not contain an antibiotic selection marker. Characteristic restriction patterns were used to verify the correctness of the isolated plasmids (FIG. 5).

### Example 3: Functional Assessment of the Antibiotic-Free System

a) Verification of long-term plasmid maintenance in the absence of antibiotics

To verify that an infA-based plasmid will be kept in the platform strain as desired, a single colony of the platform strain carrying plasmid “pQE-T7 TNFα cmR-infA” was used to inoculate 1.5 ml LB medium. The culture was incubated overnight at 37° C. with shaking at 800 rpm. The next day, 10 μl of the overnight culture were taken to inoculate another 1.5 ml LB medium. The culture was again incubated at 37° C. until the next morning with shaking at 800 rpm. This procedure was repeated for five days. A small aliquot from the last overnight culture was streaked out onto an LB agar plate and incubated at 37° C. overnight to obtain single colonies.

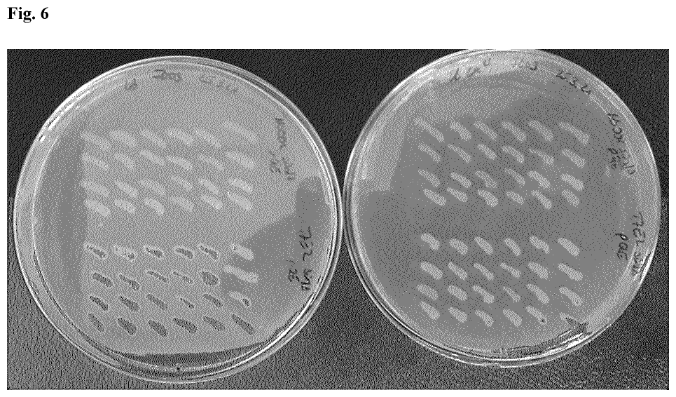

Twenty-four single colonies for both platform strains were streaked out on two LB agar plates, one conditioned with 15 μg/ml chloramphenicol and the other one without the addition of antibiotics. The plates were incubated overnight at 37° C. All clones grew on both plates confirming that plasmid “pQE-T7 TNFα cmR-infA” was maintained in all clones although the cells were grown seven days without the addition of chloramphenicol (FIG. 6). This result demonstrates an effective maintenance of the plasmid during long-term passaging without the use of antibiotics.
b) Assessment offermentation using the infA platform strain and verification of plasmid maintenance post-fermentation

To demonstrate plasmid maintenance during fermentation, the platform strain E. coli “T7E2 ΔinfA::FRT ΔrecAX::FRT-araC-araBp-infA” was used to express TNFα from the vector “pQE-T7 TNFα infA”. As a control, the infA wild-type strain E. coli T7E2 was used to express TNFα from the vector “pQE-T7 TNFα cmR”, and cultivated under continuous chloramphenicol selection pressure.

For expression, each of the aforementioned bacterial strains were inoculated into 5 ml of LB medium (supplemented with 30 μg/ml chloramphenicol for the infA wild-type strain) and grown overnight at 37° C. with 250 rpm. The following day, the overnight cultures were each inoculated into 50 ml of fresh LB medium (with 30 μg/ml chloramphenicol for the infA wild-type strain) in 250 ml baffled shake flasks to an optical density at 600 nm (OD600) of 0.1. The cultures were incubated at 37° C. with 250 rpm until an OD600 of approximately 0.5 was reached. At this point, culture samples were taken for analysis on an SDS PAGE gel. The cultures were then induced with isopropyl-β-D-thiogalactopyranosid (IPTG) to a final concentration of 1 mM. The fermentation was continued under the same conditions (37° C., 250 rpm) for a total of 48 hours. Culture samples for analysis via SDS PAGE were taken at the following time points post-induction: 1, 2, 3, 6, 24 and 48 hours.

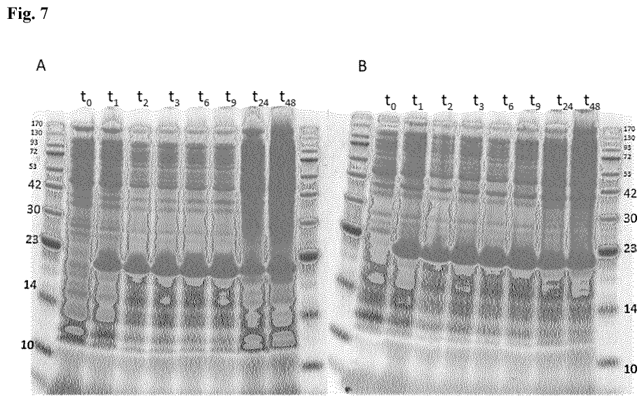

The culture samples taken for SDS PAGE analysis were standardized to 1 ml of 1 OD600 culture. The culture was pelleted, resuspended in reducing NuPAGE™ LDS Sample Buffer, heated to 70° C. for 10 minutes and then loaded on a NuPAGE™ 12% Bis-Tris gel. The samples on each gel were flanked with the protein marker PM2500 ExcelBand™. The gels were run in MES SDS Running Buffer with NuPAGE™ Antioxidant at 200 V for 35 minutes, then stained using SimplyBlue™ SafeStain. Expression of TNFα (18.8 kDa) can be seen as a thick band in all time points after induction in both strains (FIG. 7).

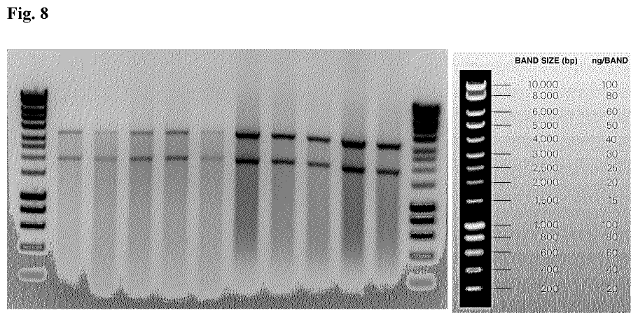

After 48 h of expression, a glycerol stock was made from the platform strain E. coli “T7E2 ΔinfA::FRT ΔrecAX::FRT-araC-araBp-infA” to analyze maintenance the plasmid “pQE-T7 TNFα infA” after two days of expression. The glycerol stock was prepared by mixing equal proportions of culture with 50% glycerol, and then stored at −80° C. For analysis, the glycerol stock was streaked on LB agar and incubated at 37° C. overnight. Ten isolated colonies were inoculated in LB medium and grown overnight at 37° C. with 800 rpm. Plasmid DNA was isolated and analyzed with restriction enzymes. All analysed colonies maintained the plasmid (FIG. 8).

### Example 4: Preparation of a Platform Strain with Arabinose-Inducible secY Gene

a) Insertion of a “cmR-araC-araBp-secY” cassette into the recAXgenomic locus to obtain an arabinose-inducible copy of the secYgene

To prove that other essential genes (besides infA) can be used for the plasmid maintenance strategy described above, another platform strain was prepared based on E. coli strain HS996. This time the essential gene secY was set under the control of the arabinose-inducible promoter. The insertion site in the genome was kept identical. For this proof-of-concept the cmR marker used for the insertion of the DNA fragment (SEQ ID NO: 18) into the recAX locus was not removed. The strain modification was performed by recombineering as described above.
b) Replacement of the endogenous secY by an FRT-flanked selection marker and removal of the marker gene by FLP recombination.

The secY gene is the second last gene of a large operon rplNXE-rpsNH-rplFR-rpsE-rpmD-rplO-secY-rpmJ which encodes several ribosomal proteins. All of these genes with the exception of rpmJ are declared “essential” according to PEC database (Profiling of E. coli Chromosome; https://shigen.nig.ac.jp/ecoli/pec/). The secY part of the operon was replaced by an FRT-flanked zeocin resistance marker gene. The selection cassette (SEQ ID NO: 19) was amplified using the primers secY-primer up (SEQ ID NO: 20) and secY-primer down (SEQ ID NO: 21), which were designed to add 50 bp long homology arms for rpmJ and rplO to the fragment during the PCR reaction. The selection marker cassette was inserted by Red/ET recombination as described above. During the recombination step the medium was supplemented with 0.4% L-arabinose. The selection marker was subsequently removed by plasmid 707-FLPe as described above.

The obtained platform strain is defined as E. coli “HS996 ΔsecY::FRT ΔrecAX::cmR-araC-araBp-secY”.

### Example 5: Preparation of an Expression Plasmid “pQE-T7 TNFα infAp2-secY” with secY Under the Control of the infA Promoter

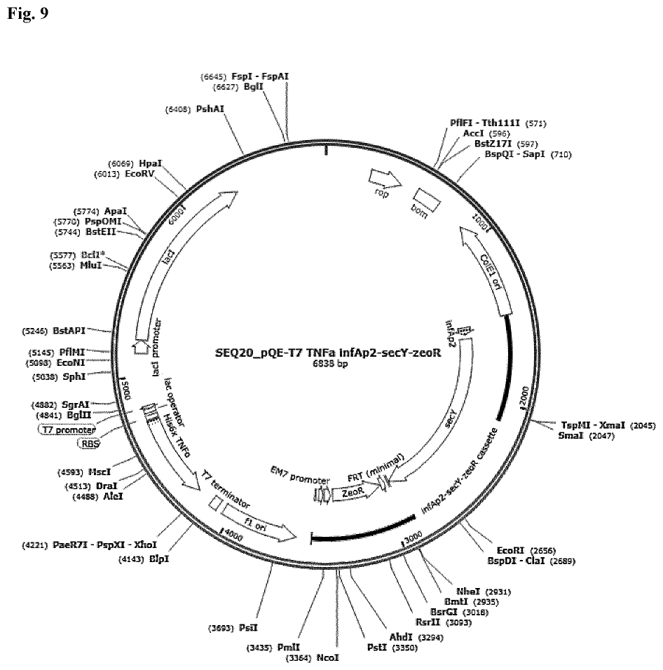

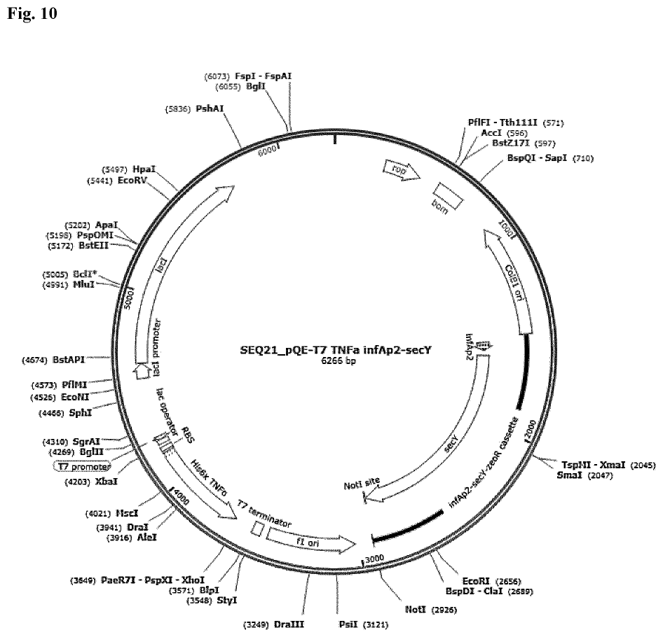

The “cmR-infA” part of the plasmid “pQE-T7 TNFα cmR-infA” was replaced by a secY open reading frame linked to a zeoR resistance marker gene. The zeocin resistance marker gene was flanked by NotI sites. To test whether a promoter other than the natively associated promoter can be used for this technology, the infA promoter was kept to drive expression of the secY gene. Recombineering proficient E. coli strain GB08-red was again used for the plasmid modification. The fifty base pair long homology arms for the recombination with plasmid “pQE-T7 TNFα cmR-infA” were chosen to replace the “cmR-infA” cassette in the original plasmid by “zeoR-secY”. The obtained plasmid “pQE-T7 TNFα infAp2-secY-zeoR” (FIG. 9, SEQ ID NO: 22) was digested with restriction enzyme NotI and subsequently religated to obtain a test plasmid without any resistance marker gene. This test plasmid was named “pQE-T7 TNFα infAp2-secY” (FIG. 10, SEQ ID NO: 23).

### Example 6: Confirmation of Plasmid Maintenance Based on infAp2-secY

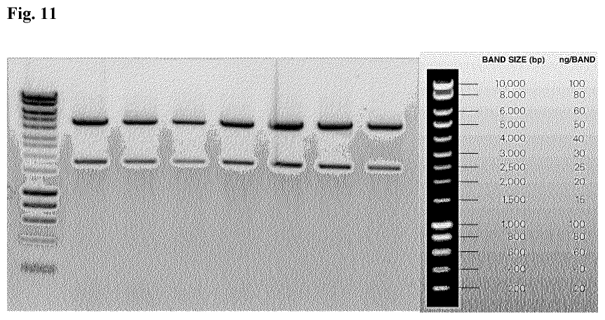

Electrocompetent cells were prepared from the platform strain E. coli “HS996 ΔsecY::FRT ΔrecAX::cmR-araC-araBp-secY” as described in the manual of the “Quick & Easy E. co/i Gene Deletion Kit” (Gene Bridges, Cat. No. K006). Approximately 10 ng of the plasmid “pQE-T7 TNFα infAp2-secY” was electroporated into the cells of the platform strains. The cells were resuspended after electroporation in 1 ml LB medium (without antibiotics or L-arabinose) and incubated for two hours at 37° C. with shaking at 800 rpm. After this recovery phase the cells were streaked out on LB agar plates (without antibiotics or L-arabinose) and incubated overnight at 37° C. About fifty colonies were visible on the plates the next day. Seven colonies were inoculated in LB medium without the addition of antibiotics and grown overnight at 37° C. with 800 rpm. Plasmid DNA was isolated from these clones and analysed with restriction enzymes to confirm the presence of the plasmid in the cells. All analysed colonies display the expected restriction pattern (FIG. 11).

No antibiotic selection was used to insert or keep the plasmid in the cells. Cells are only viable in the presence of a plasmid that carries a copy of the secY gene driven by an appropriate promoter, in this case infAp2. The plasmid does not carry any antibiotic resistance marker genes.

### CITED LITERATURE

Zhang, Y., Muyrers, J. P., Testa, G. and Stewart, A. F. (2000). “DNA cloning by homologous recombination in Escherichia coli.” Nature Biotechnology 18: 1314-7 https://doi.org/10.1038/82449;
Murphy, K. C., Campellone, K. G. and Poteete, A. R. (2000). “PCR-mediated gene replacement in Escherichia coli.” Gene 246: 321-30. https://doi.org/10.1016/S0378-1119(00)00071-8;
Fu, J., Teucher, M., Anastassiadis, K., Skarnes, W. and Stewart, A. F. (2010). “A recombineering pipeline to make conditional targeting constructs.” Methods in Enzymology 477: 125-144. https://doi.org/10.1016/S0076-6879(10)77008-7;
Szpirer, C. Y., and Milinkovitch, M. C. (2005). “Separate-component-stabilization system for protein and DNA production without the use of antibiotics.” Biotechniques 38, 775-781. https://doi.org/10.2144/05385RR02;
Cranenburgh, R. M., Hanak, J. A., Williams, S. G., and Sherratt, D. J. (2001). “Escherichia coli strains that allow antibiotic-free plasmid selection and maintenance by repressor titration.” Nucleic Acids Research 29, E26. https://doi.org/10.1093/nar/29.5.e26;
Mairhofer, J., Cserjan-Puschmann, M., Striedner, G., Nobauer, K., Razzazi-Fazeli, E., and Grabherr, R. (2010). Marker-free plasmids for gene therapeutic applications—Lack of antibiotic resistance gene substantially improves manufacturing process.” Journal Biotechnology. 146, 130-137. https://doi.org/10.1016/j.jbiotec.2010.01.025;
Fiedler, M., and Skerra, A. (2001). “proBA complementation of an auxotrophic E. coli strain improves plasmid stability and expression yield during fermenter production of a recombinant antibody fragment.” Gene 274, 111-118. https://doi.org/10.1016/S0378-1119(01)00629-1;
WO2003/000881;
WO2017/097383;
WO2018/083116;
Brautaset, T.,Lale, R., and Valla, S. (2009). “positively regulated bacterial expression systems”., Microbial Biotechnology 2(1), 15-30. https://doi.org/10.1111/j.1751-7915.2008.00048.x
Shimada, T., Yamazaki, Y., Tanaka, K., and Ishihama, A. (2014), PLoS ONE 9(3): e90447. https://doi.org/10.1371/journal.pone.0090447.

1-19. (canceled)

20. A recombinant bacterial cell comprising in its genome
(i) at least one endogenous essential gene which is inactivated, and
(ii) at least one introduced copy of the said at least one essential gene, wherein said introduced copy is operatively linked to a heterologous expression control sequence being inducible by an inducer molecule, such that the expression of said essential gene in the recombinant bacterial cell is dependent on the presence of said inducer molecule.

21. The recombinant bacterial cell of claim 20, wherein said bacterial cell is an E. coli cell.

22. The recombinant bacterial cell of claim 20, wherein said at least one essential gene is a gene required for cell growth and/or viability.

23. The recombinant bacterial cell of claim 22, wherein the at least one essential gene is a gene required for cell growth and/or viability is selected from the group consisting of: infA, infC//F-3, dnaJ, dnaK, era, frr, ftsL, ftsN, ftsZ, grpE, mopA, mopB, msbA, nusG, parC, rpsB, secY and trmA.

24. The recombinant bacterial cell of claim 23, wherein said at least one essential gene is infA or secY.

25. The recombinant bacterial cell of claim 20, wherein the bacterial genome lacks any gene being capable of functionally complementing the at least one essential gene in the absence of the inducer molecule.

26. The recombinant bacterial cell of claim 20, wherein said bacterial cell comprises an expression plasmid which comprises at least one plasmid copy of the at least one essential gene, wherein said plasmid copy is under the control of the expression control sequence which is biologically active in said bacterial cell.

27. The recombinant bacterial cell of claim 26, wherein said expression control sequence which is biologically active is the expression control sequence which is natively associated with the at least one endogenous essential gene.

28. The recombinant bacterial cell of claim 26, wherein said expression plasmid comprises at least one further nucleic acid of interest, which is required for the recombinant manufacture of a compound of interest in said recombinant bacterial cell.

29. The recombinant bacterial cell of claim 20, wherein said inducer molecule is selected from the group consisting of: arabinose, rhamnose, xylose, sucrose, tetracycline, anhydrotetracycline, and IPTG.

30. A method for generating the recombinant bacterial cell of claim 20 comprising the steps of:
(a) introducing into the genome of a bacterial cell at least one copy of at least one essential gene being operatively linked to a heterologous expression control sequence being inducible by an inducer molecule; and
(b) inactivating the at least one endogenous essential gene in the genome of the bacterial cell, such that the expression of said essential gene in the recombinant bacterial cell is dependent on the presence of inducer molecule.

31. The method of claim 30, wherein the bacterial cell is cultivated after step (b) in the presence of the inducer molecule.

32. The method of claim 31, wherein the inducer molecule is selected from the group consisting of: arabinose, rhamnose, xylose, sucrose, tetracycline, anhydrotetracycline, and IPTG.

33. A method for recombinant manufacture of a compound of interest comprising the steps of:
(a) introducing into a recombinant bacterial cell of claim 20, an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.

34. The method of claim 33, wherein said expression plasmid comprises at least one further nucleic acid of interest which is required for the recombinant manufacture of a compound of interest in said recombinant bacterial cell.

35. The method of claim 33, wherein said method further comprises:
(c) obtaining the compound of interest manufactured by the recombinant bacterial cell.

36. The method of claim 20, wherein said expression control sequence which is biologically active is the expression control sequence which is natively associated with the at least one endogenous essential gene.

37. A method for the recombinant manufacture of a compound of interest, the method comprising using the recombinant bacterial cell of claim 26.

38. A kit for recombinant manufacture of a compound of interest comprising:
(i) a recombinant bacterial cell of claim 20, and
(ii) an expression plasmid which comprises at least one plasmid copy of the at least one essential gene, wherein said plasmid copy is under the control of the expression control sequence which is biologically active in said bacterial cell.

39. The kit of claim 38, wherein said expression control sequence which is biologically active is the expression control sequence which is natively associated with the at least one endogenous essential gene.

40. The kit of claim 38, wherein said expression plasmid comprises at least one further nucleic acid of interest which is required for the recombinant manufacture of a compound of interest in said recombinant bacterial cell which is required for the recombinant manufacture of a compound of interest in said recombinant bacterial cell.
